(19) United States
(12) Patent Application Publication
(10) Pub. No.: US 2008/0193508 A1
(43) Pub. Date: Aug. 14, 2008

(54) Local anti-infective agent for treatment of nail fungal infections

(75) Inventors: David M. Cohen, Lauderdale by the Sea, FL (US); Eugene R. Cooper, Berwyn, PA (US)

Correspondence Address: PAUL D. BIANCO;Fleit Gibbons Gutman Bongini & Bianco PL, 21355 EAST DIXIE HIGHWAY, SUITE 115, MIAMI, FL, 33180, US

(73) Assignee: DermWoRX, Inc., Hollywood, FL (US)

(21) Appl. No.: 12/028,188

(22) Filed: Feb. 8, 2008

Related U.S. Application Data
(60) Provisional application No. 60/888,825, filed on Feb. 8, 2007.

(51) Int. Cl. A61K 31/195; A61K 31/20; A61K 31/19; A61K 9/70; A61P 31/00; A61K 31/135; A61K 31/192

(52) U.S. Cl. 424/447; 514/558; 514/557; 514/570; 514/655; 514/562

(57) ABSTRACT

A topical composition for treating nail fungal infections that utilizes an acidic antifungal agent with a molecular weight no greater than 170 Daltons in a formulation having a pH less than or equal to the pKa of the acidic antifungal agent plus one. Possible antifungal agents include omadine, octanoic acid, sorbic acid, hexanoic acid, and benzoic acid. The antifungal agent can be combined with a delivery system such as a lacquer, a gel, a patch, or a hydrating system. A second therapeutic agent such as a 5-fluorocystine or terbinafine can be included.

### CROSS-REFERENCE TO RELATED APPLICATIONS

This application claims the benefit of U.S. Provisional Application No. 60/888,825, filed Feb. 8, 2007.

### STATEMENT REGARDING FEDERALLY SPONSORED RESEARCH OR DEVELOPMENT

Not Applicable

### THE NAMES OF PARTIES TO A JOINT RESEARCH AGREEMENT

Not Applicable

### INCORPORATION-BY-REFERENCE OF MATERIAL SUBMITTED ON A COMPACT DISC

Not Applicable

### BACKGROUND OF THE INVENTION

1. Field of the Invention

The invention relates to topical antifungal treatments for toe and finger nails, and, in particular, to topical antifungal treatments for dermatophytes, fungal agents, yeast, and non-dermatophyte molds.

2. Description of the Related Art

Nail infections can be caused by a number of agents, many known, but some unknown at this time. These agents include molds, fungi and yeast organisms. For onychomycosis, some of the identified organisms include C. albicans, C. parappsilosis and Scopulariopsis brevicaulis. Other etiologic agents of oncychomycosis include Aspergillus flavus, A. candidus, A. fumigatus, A. sydowi, A. terreus, and other aspergillus species, Cephalosporum species and Fusrium oxysporum. Onychomycosis is associated with chronic paronychia where active invasion of the nail plate is less frequent. Tinea ungium is an invasive disease of the nail plate caused by a dermatophyte, most commonly T. interdigitale, T. rubrum and T. metagrophytes. Generally, tinea ungium is classified into two subtypes—leukonychia mycotica (superficial white onychomycosis, SWO) and invasive sublingual onychomycosis (commonly called ringworm of the nail). In leukonychia mycotica, the nail is invaded from the top exhibiting pitting or infected patches on the surface of the nail. This type of infection is almost always produced by T. mentagrophytes.

Onychomycosis has long been recognized as one of the most difficult fungal infections to treat. The lengthy period over which the nail takes to grow, the hardness of the nail plate, the location of the infectious process between the nail bed and plate are major factors for difficulty in treatment. Current treatments for these conditions are primarily oral anti-infective agents that are lipophilic and have very large molecular weights and thus would have poor nail penetration. Examples of these agents include terbinafine, itraconazole, and fluconazole. These agents are given for months on end and are associated with systemic toxicity. In order to avoid systemic toxicities, it is important to develop methods to treat these local infections using local anti-infective agents. Such treatments can provide significant improvements to current therapies including less toxicity and potentially greater efficacy.

One currently commercially-available topical treatment of onychomycosis is sold under the trade name CICLOPIROX. CICLOPIROX has only been marginally successful and it has been shown to have poor penetration across the layers of the nail. The nail, a highly keratinized membrane, is a formidable barrier to diffusion, and the approach has been to utilize existing oral antifungal drugs in topical formulations so that they might penetrate the nail, but this approach has not worked well.

### BRIEF SUMMARY OF THE INVENTION

It is accordingly an object of the invention to provide a local anti-infective agent for treatment of nail fungal infections that overcomes the above-mentioned disadvantages of the heretofore-known devices and methods of this general type.

With the foregoing and other objects in view there is provided, in accordance with the invention, a composition for topical administration that utilizes an acidic antifungal agent with a molecular weight no greater than 170 Daltons in a formulation having a pH no more than the pKa of said acidic antifungal agent plus one. The pKa is the negative log of the acid dissociation constant. By being in a pH equal to the pKa, at least fifty percent of the acidic antifungal agent is in its free acid form. The pH of the formulation is preferably less than or equal to the pKa. The term “equal to the pKa” is meant to mean that the pH is equal to the pKa ±0.1.

Possible antifungal agents include, but are not limited to, omadine, octanoic acid, sorbic acid, hexanoic acid, and benzoic acid.

In accordance with further objects of the invention, the composition can include a delivery system for delivering the acidic antifungal agent to an infected are such as a nail, preferably a toenail. The delivery system can be a lacquer, a gel, a patch, any film-forming system, or a hydrating system. A concentration of the antifungal agent in the delivery system ranges from 0.1% to 20%, preferably from 1% to 6%.

The delivery system can include a film forming polymer for forming a film including the active ingredient on an infected area after application. The film forming polymer can be a cellulose or a cellulose derivative. The film forming polymer might be initially dissolved in an organic solvent or an aqueous solvent. Possible film forming polymers include polyvinyl alcohol and polyvinylpyrrolidone. A glycol such as glycerol and propylene glycol can be added.

When the delivery system is a patch, the patch is attached over an infected area with an adhesive. The adhesive can contain the active ingredient.

The composition can be combined with a second therapy (including an acidic antifungal agent). The second therapy (i.e. therapeutic agent or therapeutic agent system) can also be 5-fluorocystine or terbinafine.

This patent application describes formulations for enhanced topical delivery to treat nail infections also called onychomycosis and/or tinea unguium (also sometimes called ringworm of the nail). The causative agents for these conditions are a series of infective agents which encompass dermatophytes, fungal agents, yeast or non-dermatophyte molds. We describe here an invention for the use of small molecular weight substances with particular characteristics and particular formulations to treat these conditions topically.

The invention of this application includes a topical therapy utilizing antifungal agents based on the molecular properties that allows passage across the nail. Furthermore, the formulations ensure the penetration of these agents across the multiple layers that form the nail. The nail barrier is very sensitive to the size (and hence the molecular weight) of the diffusing molecule. In fact, nail diffusion is so selective for molecular weight that agents with molecular weight preferably below 170 Daltons, but not limited to such, are the best choice for topical treatment of onychomycosis. The invention encompasses using all low molecular weight (i.e. less than 170 Daltons) acidic compounds that are generally know anti-fungal agents, as effective agents to treat nail-related fungal infections.

Examples of such compounds include but are not limited to the following compounds: 1) omadine 2) low molecular weight fatty acids such as octanoic acid, sorbic acid, hexanoic acid, and 3) benzoic acid. Of particular usefulness is omadine. Neither this agent nor the other agents are currently oral therapies for fungal and dermatophytic infections. The molecular weight of omadine (pyrithione) is 127 Daltons.

It is preferred that the small molecular weight compounds covered in this patent have a solubility in water above 1 mg/ml but not limited as such. Combinations of agents with the properties discussed or with other existing therapies would also be effective for treating onychomycosis. The agents described can be formulated, but not limited to, as films, lacquers, gels, patches, hydrating systems, etc. for topical delivery and can be dosed in concentrations of 50% and below but not necessarily limited as such. They can be incorporated into polymer films and applied directly to the nail. For cosmetic purposes, agents may be added to mask the unpleasant appearance of the infected nail. These films can be similar to traditional fingernail films used to color the nail. They can contain any type of film forming polymer such as celluloses or cellulosic derivatives and can be formed from organic solvents and/or solvents containing water.

Other features that are considered as characteristic for the invention are set forth in the appended claims.

Although the invention is illustrated and described herein as embodied in a local anti-infective gent for treatment of nail fungal infections, it is nevertheless not intended to be limited to the details shown because various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

Not Applicable

### DETAILED DESCRIPTION OF THE INVENTION

Referring now to the examples described below, there detailed examples of formulations containing omadine or fatty acids are solutions or dispersions containing water at pH equal to or below the pKa (acid dissociation constant), organic solvents, and a polymer. All percentages used in the application are weight percentages unless otherwise noted.

### Example 1

A formulation was prepared by mixing the following components:

10% omadine at pH 4.7

admix isopropyl alcohol and Klucel to produce a final concentration of omadine of 1%.

### Example 2

A formulation was prepared by mixing the following components:

10% omadine at pH 4.7

admix isopropyl alcohol and Klucel to produce a final concentration of omadine of 2.5%.

### Example 3

A formulation was prepared by mixing the following components:

Hexanoic acid dispersed in water at pH 3, and

admix with 3% Klucel to have a 1% hexanoic acid dispersed in the gel.

The previous examples are not limiting. The treatment according to the invention which is to be applied locally to the nail to treat and/or prevent onychomycosis, may be formulated to treat a particular organism or group of organisms that may be characterized in the future.

### Example 4

A study determines the penetration of sodium omadine in human nails. The study analyzes the Penetration of Hydroxypyridine-2-Thione Sodium into human fingernails.

The experiment studies the penetration of stock solutions.

Sodium Omadine (NaOM) 98% was obtained from Acros Organics, a division of Global Fisher Scientific.

Stock Solution #1. Dissolve 100 mg of NaOM in 100 ml of purified water (1.0 mg/ml).

Stock Solution #2. Dilute 2 ml of Stock Solution #1 to 100 ml of purified water (20 μg/ml).

Standard Solution #1 (2.50 μg/ml). Dilute 12.5 ml of Stock Solution #2 to 100 ml with purified water.

Standard Solution #2 (5.00 μg/ml). Dilute 25 ml of Stock Solution #2 to 100 ml with purified water.

Standard Solution #3 (7.50 μg/ml). Dilute 37.5 ml of Stock Solution #2 to 100 ml with purified water.

Standard Solution #4 (10.0 μg/ml). Dilute 50 ml of Stock Solution #2 to 100 ml with purified water.

Standard Solution #5 (20.0 μg/ml). No dilution necessary, same as Stock Solution #2.

Nail clips, pooled from six human volunteers are cut into small pieces of approximately 0.5-1 mm×2-3 mm in size. The nail clips, 0.1 g, are placed in each of the above solutions at room temperature and incubated for twenty-four hours. After incubation, the clips are washed quickly three times with purified water to remove any residual solution on the nail surface. The clips are then incubated in 1 ml of purified water for twenty-four hours at room temperature to extract the NaOM absorbed into the nail bed (Extracting Solution).

A standard curve of NaOM is made by measuring the absorption at 318 nm for a 5001 sample containing 0.25, 0.50, 0.75, 1.0, and 2.0 μg of NaOM. Dilute 50 μl of each Standard Solution (#1-#5) to 500 μl with 450 μl of 1 mM Cupric Chloride (CuCl₂). The standard solutions are prepared fresh and incubated at room temperature for fifteen minutes prior to spectrophotometric determination.

The amount of NAOM in the Extracting Solution is determined at 318 nm by mixing 50 μl aliquot with 450 μl of 1 mM CuCl₂ and incubating at room temperature for 15 minutes prior to spectrophotometric determination. The concentration of the NaOM adsorbed into the nail bed is determined from the standard curve.

The standard curve at the concentrations between 0.25-5 μg/500 μl was linear. The amount of NaOM in each sample was calculated and presented in the table:

| | NaOM | NaOM | |
|---|---|---|---|
| Wk Sol Conc | in 50 μl sample | conc in sample | NaOM in 0.1 g nail |
| 0.1 mg/ml | 1.33 μg | 27 μg/ml | 3.99 μg |
| 0.05 mg/ml | 0.70 μg | 14 μg/ml | 2.10 μg |
| 0.01 mg/ml | 0.34 μg | 7 μg/ml | 1.02 μg |

### Example 5

An experiment was conducted to determine the penetration of hydroxypridine-2-thione zinc (PT) into human fingernail.

The experimental procedure including the following steps. Nail clips pooled from six volunteers were minced into small pieces of 0.5-1 mm×2-3 mm. PT was dissolved in 100% DMSO at a concentration of 2 mg/ml to form a PT stock solution. PT working solution was made by mixing 300 μl of the PT stock solution with 700 μl of water. The final concentration was 0.6 mg/ml (30% DMSO). The nail clips (0.1 g) were incubated in 1 ml of PT working solution overnight at room temperature. After incubation, the clips were washed with water three times in a spin column. The clips were then incubated in 200 μl of 30% DMSO to extract PT for 4 hours at room temperature. A sample of the extracting solution was collected. A standard curve of PT was established by measuring the absorption at 318 mm in 500 μl samples containing 0.25, 0.5, 0.75, 1, and 2 μg PT. PT in 30% DMSO (50 μl) was mixed with 450 μl of 1 mM CuCl₂ and incubated at room temperature for 5 min. The amount of PT in the extracting solution was measure by mixing 50 μl of sample with 450 μl of mM CuCl₂ at 318 nm.

The following results were obtained. The standard curve at the concentrations between 0.25-2 μg/500 μl was linear. The absorption of nail extract was 0.111, and was calculated to be 1.15 ug/50 ul, or 23 ug/ml.

The following conclusion was made based on the results. As demonstrated in the results section, there is significant penetration of Hydroxypyridine-2-Thione Zinc into fingernails after twelve hours or less incubation time.

We claim:

1. A composition for topical administration, comprising an acidic antifungal agent with a molecular weight no greater than 170 Daltons in a formulation having a pH no more than a pKa of said acidic antifungal agent plus one.

2. The composition according to claim 1, wherein said acidic antifungal agent is selected from the group consisting of omadine, octanoic acid, sorbic acid, hexanoic acid, and benzoic acid.

3. The composition according to claim 1, wherein said pH of said formulation is equal to said pKa.

4. The composition according to claim 1, wherein said pH of said formulation is no less than said pKa minus one.

5. The composition according to claim 1, further comprising a delivery system for delivering said acidic antifungal agent to a nail.

6. The composition according to claim 5, wherein said delivery system is selected from the group consisting of a lacquer, a gel, a patch, and a hydrating system.

7. The composition according to claim 5, wherein a concentration of said antifungal agent in said delivery system ranges from 0.1% to 20%.

8. The composition according to claim 7, wherein said concentration of said antifungal agent in said delivery system ranges from 1% to 6%.

9. The composition according to claim 5, wherein said delivery system includes a film forming polymer for forming a film on an infected area after application.

10. The composition according to claim 9, wherein said film forming polymer is selected from the group consisting of a cellulose and a cellulose derivative.

11. The composition according to claim 9, wherein said film forming polymer is initially dissolved in an organic solvent.

12. The composition according to claim 9, wherein said film forming polymer is initially dissolved in an aqueous solvent.

13. The composition according to claim 9, wherein said film forming polymer is selected from the group consisting of polyvinyl alcohol and polyvinylpyrrolidone.

14. The composition according to claim 1, further comprising a glycol.

15. The composition according to claim 14, wherein said glycol is selected from the group consisting of glycerol and propylene glycol.

16. The composition according to claim 9, further comprising a glycol.

17. The composition according to claim 16, wherein said glycol is selected from the group consisting of glycerol and propylene glycol.

18. The composition according to claim 7, wherein:
said delivery system is a patch;
said patch has an adhesive to attach said patch over an infected area; and
said active ingredient is contained in said adhesive of said patch.

19. The composition according to claim 18, wherein the infected area is a toenail.

20. The composition according to claim 1, which further comprises a second therapy capable of treating fungal infections simultaneously with the composition according to claim 1.

21. The composition according to claim 20, wherein said second therapy is 5-fluorocystine.

22. The method according to claim 20, wherein the second therapy is terbinafine.

23. A method of treating onychomycosis, which comprises treating a toenail with a composition according to claim 1.
